(19) United States
(12) Patent Application Publication
(10) Pub. No.: US 2019/0015307 A1
(43) Pub. Date: Jan. 17, 2019

(54) INORGANIC PIGMENTS COMPOSED OF SPECIAL SPHERICAL COMPOSITE PARTICLES AS BOOSTER FOR SUN PROTECTION

(71) Applicant: MERCK PATENT GMBH, DARMSTADT (DE)

(72) Inventors: Michael TERMER, Hofheim (DE); Frank PFLUECKER, Darmstadt (DE); Lilia HEIDER, Gernsheim (DE); Alexandra AXT-HEIDEMANN, Alsbach-Haehnlein (DE); Jutta ZUR LAGE, Darmstadt (DE); Sherry WU, Taipei (TW)

(73) Assignee: MERCK PATENT GMBH, DARMSTADT (DE)

(21) Appl. No.: 16/067,245

(22) PCT Filed: Dec. 8, 2016
(86) PCT No.: PCT/EP2016/002068

§ 371 Date: Jun. 29, 2018

(30) Foreign Application Priority Data
Dec. 31, 2015 (CN) 201511021412.4

(51) Int. Cl. A61K 8/29; A61K 8/28; A61K 8/27; A61K 8/25; A61K 8/02; A61K 8/06; A61K 8/04; A61Q 17/04

(52) U.S. Cl. CPC A61K 8/29; A61K 8/28; A61K 8/27; A61K 8/25; A61K 8/025; A61K 2800/651; A61K 8/064; A61K 8/042; A61Q 17/04; A61K 2800/43; A61K 2800/621; A61K 8/062

(57) ABSTRACT

The present invention is directed to the use of at least one kind of inorganic pigments composed of a spherical composite particle comprising an inorganic UV filter to enhance the sun protection of a cosmetic formulation.

### FIELD OF THE INVENTION

The present invention is directed to the use of at least one kind of inorganic pigments composed of a spherical composite particle comprising at least one metal oxide to enhance the sun protection of a cosmetic formulation.

### BACKGROUND OF THE INVENTION

Skin protection against UV radiation is a necessity as the connection between serious skin conditions and a changed lifestyle has become more obvious in the last years. This has led formulation developer to use UV filters not only in sunscreens, but also in face care products.

Sunscreen formulations with high sun protection factors (SPFs) often rely on a mixture of organic and inorganic UV filters.

There is an on-going development of a wide range of emollients, antioxidants, film former or hollow organic spheres to improve the UV filters and boost the SPF of cosmetic formulations.

Different emollients can help to get a better spreadability of the emulsion on the skin and therefore improve the overall SPF of the cosmetic formulation. Antioxidants on the other hand can help to stabilize the UV filters and their degradation.

Film former or water thickener can lock the inorganic UV filters in the emulsion and prevent re-agglomeration, which would lead to a lower SPF value of the cosmetic formulation.

The use of organic UV filter leads to a more greasy and sticky skin feel of the formulations. The possibility to increase the SPF with inorganic nanoparticles e.g. inorganic UV filters, has a limit were either the formulation is cosmetically not acceptable (whitening effect, harsh or dry skin feel) or the stability of the cosmetic formulation is strongly negatively impacted.

The commercial product NS Boost from NextSTep Laboratories is a ceramic-like platelet of mica and silica that boosts the effectiveness of sunscreens. It is further described for this commercial product that it effectively coats the skin and helps to spread sunscreens evenly due to its particular shape. The boost is described for particulate or organic sunscreens.

WO 2014/097972 describes a composition comprising at least one organic UV filter, a spherical composite particle with a mean size of 1 to 20 μm comprising a matrix and an inorganic UV filter, boron nitride and spherical porous silica particles. Such a composition is described as having a good skin sensitization and a high SPF. It is further described that the spherical silica particles act as agent to increase the sun protection factor (SPF). No further explanations were given to the role of the other ingredients of the specific composition.

WO 2012/104161 describes a composition comprising an UV filter and non-spherical particles comprising an inorganic UV filter together with spherical composite particles comprising an inorganic UV filter, which has a higher SPF and a better cosmeticity than a composition comprising the UV filter, and the spherical composite particle.

The enhancement of the sun protection factor (SPF) by glass microspheres is known from EP 1506772.

However, there continues to be a demand for the development of substances, which enhance the sun protection factor of a cosmetic formulation and additionally preserve or enhance a good skin feeling of the cosmetic formulation.

The object of the invention is therefore to provide substances to be incorporated in a cosmetic formulation, which are able to enhance the sun protection of a cosmetic formulation and to preserve or enhance a good skin feeling of the cosmetic formulation.

Surprisingly it was found that inorganic pigments composed of a spherical composite particle comprising at least one metal oxide fulfil such demands.

Inorganic pigments composed of a spherical composite particle comprising at least one metal oxide are known in the art.

EP 2316891 A1 describes such materials as fillers.

Fillers can be regarded as a special form of pigments. In fillers, it is not the “colouring” function that is in the foreground. Instead, factors such as an increase in mechanical stability, abrasion resistance, weather stability or also production costs are crucial for use of industrial fillers.

Fillers are also widely used in cosmetic formulations. For example, powders may comprise up to 50% of fillers, based on the final formulation. Typical values are 10-15% of fillers in lipsticks and 2-6% of fillers in emulsions. Cosmetic fillers have a wide variety of functions: in foundations, they prevent an undesired greasy sheen on the skin due to the so-called matting effect, while in powders they help, for example, to improve the pouring behaviour or the skin application properties.

### SUMMARY OF THE INVENTION

The invention relates to the use of at least one kind of inorganic pigments composed of a spherical composite particle comprising at least one metal oxide to enhance the sun protection of a cosmetic formulation comprising at least one organic UV filter and/or at least one inorganic UV filter.

### DETAILED DESCRIPTION OF THE INVENTION

The sun protection of a cosmetic formulation can be classified through its sun protection factor (SPF). This factor e.g. classifies the efficiency of cosmetic formulations comprising UV filters. The SPF is expressed mathematically as the ratio of the irradiation time necessary to reach the erythema-forming threshold with the UV filter to the time necessary to reach the erythema-forming threshold without UV filter.

Many methods are known for the measurement of the SPF in vivo. In Europe, the measurement according to ISO 244444:2010 is communicated as standard. The US established a method called FDA-Method (FDA=Food and Drug Administration). Similar methods exist in Japan and Australia. The main difference between these methods is the used light source and the number of test persons. The SPF is evaluated in vivo mostly according to the international method published by Colipa, CTFA SA, JCIA, May 2006.

Many methods are also known for the measurement of the SPF in vitro. The method used herein for the documentation of the SPF boost in vitro is based on the method according to ISO 24443:2012 and contains the measurement of the diffuse transmission of the cosmetic formulation.

The measurement is done in the UV spectrum between 290 nm and 400 nm. The sample is applied on a UV-permeable plate and is irradiated with a specific UV light source. The transmittance of the cosmetic formulation is detected via a UV spectrophotometer. The in vitro SPF is then calculated using the integrated software in the spectrophotometer. Preferable spectrophotometers are described in the examples.

In principle, there is no limitation to the type of cosmetic formulation comprising at least one organic UV filter and/or at least one inorganic UV filter, which sun protection, is enhanced according to the invention.

Cosmetic formulations can be in the form of simple or complex emulsions (O/W, W/O, W/Si, O/W/O or W/O/W), such as creams, milks or also gels, gel creams, powders and solid sticks, and they may, if desired, be formulated as aerosols and be in the form of foams or sprays.

The cosmetic formulation may comprise cosmetic adjuvants that are usually used in this type of formulation, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

Preferred cosmetic formulations according to the invention are emulsions or gels, particular preferably water-in-oil emulsions, water-in-silicone emulsions, gels or oil-in-water emulsions.

An emulsion is a mixture of two or more liquid phases that are normally immiscible. In an emulsion, one liquid (the dispersed phase) is dispersed in the other (the continuous phase). Emulsions comprise, for example, fats, oils, waxes or other fatty substances in one phase, as well as water, polyols or preservatives in the aqueous phase. Additionally an emulsifier can be added in the oil or water phase.

There exists different types of emulsions, which are classified by the used emulsifier.

A water-in-oil emulsion is an emulsion wherein an aqueous phase is the dispersed phase and a lipid phase (or synonymously an oil phase) is the external phase.

A water-in silicone emulsion is an emulsion wherein an aqueous phase is the dispersed phase and silicones build the external lipid phase. Generally, an emulsion is a water-in-silicone emulsion when a silicone-based emulsifier is used. Furthermore, it is preferred to use silicone oils.

An oil-in-water emulsion is an emulsion wherein the lipid phase is the dispersed phase and the aqueous phase is the dispersion medium.

A gel is a disperse system consisting of at least two components. One component forms a sponge-like, three-dimensional network whose pores are filled by a liquid. The liquid component is thereby immobilized in this network. A hydrogel can contain over 90% water with a network of natural or synthetic polymer chains that are hydrophilic.

The cosmetic formulation may also be in the form of an alcoholic gel, which comprises one, or more lower alcohols or polyols, such as ethanol, propylene glycol or glycerine, and a thickener, such as siliceous earth. The oily-alcoholic gels also comprise natural or synthetic oil.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerine, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

A suitable lipid phase of the cosmetic formulation comprising at least one organic UV filter and/or at least one inorganic UV filter is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acids and saturated and/or unsaturated, branched and/or unbranched alcohols having a chain length of 3 to 30 C atoms. Ester oils of this type can then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The lipid phase may advantageously be selected from the group of branched and unbranched hydrocarbons and hydrocarbon waxes, silicone oils, dialkyl ethers, or the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may advantageously be selected, for example, from the group of synthetic, semi-synthetic and natural oils, for example olive oil, sunflower oil, soya oil, peanut oil, rapeseed oil, almond oil, palm oil, coconut oil, palm kernel oil and the like.

The lipid phase may contain cosmetic oils (synthetic and/or natural), organic solvents, both non-ionic and ionic (inter alia so-called ionic liquids), lipophilic or amphiphilic thickeners, softeners, humectants, opacifiers, stabilisers, silicone oils and silicone oil derivatives, antifoam agents, perfume, preservatives, anionic, cationic, non-ionic, zwitterionic surfactants, cosmetic active ingredients, fillers, polymers, propellant gases, acids and/or lyes, and any desired substance which is generally used in cosmetics.

Fat-containing substances can be oils or waxes or mixtures thereof. The term oil means substances and compounds, which are liquid at room temperature. The term waxes means substances and compounds which have a solid or semisolid consistency and whose melting point is >35° C.

The term oils includes mineral oils (paraffin oils), vegetable oils (such as, for example, jojoba oil), synthetic oils, such as, for example, perhydrosqualenes, fatty alcohols, fatty acids or fatty acid esters, such as, for example, the C12-C15 alkyl benzoate commercially available under the trade name “Witconol TN” from Witco, octyl palmitate, isopropyl lanolate and triglycerides, including capric/caprylic acid triglycerides, silicone oils (cyclomethicone and polydimethylsiloxanes, or PDMS) or fluorinated oils, and polyalkylenes.

Wax constituents of a cosmetic formulation can be, for example, paraffin wax, carnauba wax, beeswax, or hydrogenated castor oil.

The possible organic solvents include, inter alia, lower alcohols and polyols. Polyols can be selected, for example, from the following substances/classes of substance: glycerine, glycol ethers, ethylene glycol, propylene glycol, butylene glycol, dipropylene glycol, diethylene glycol.

The lipid phase is advantageously selected from the group of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethyl-hexyl isostearate, mixtures of C₁₂₋₁₅-alkyl benzoate and isotridecyl isonon-anoate, as well as mixtures of C₁₂₋₁₅-alkyl benzoate, 2-ethylhexyl isostearate and isotridecyl isononanoate.

Furthermore, the lipid phase may also advantageously have a content of cyclic or linear silicone oils or consist entirely of oils of this type, especially preferably in water-in-silicone emulsions.

A suitable silicone oil is cyclomethicone (octamethylcyclotetrasiloxane). However, it is also advantageous for the purposes of the present invention to use other silicone oils, for example hexamethylcyclotrisiloxane, polydimethyl-siloxane, poly(methylphenylsiloxane).

Also particularly advantageous are mixtures of cyclomethicone and iso-tridecyl isononanoate and of cyclomethicone and 2-ethylhexyl isostearate.

The aqueous phase advantageously comprises alcohols, diols or polyols having a low carbon number, and ethers thereof, preferably ethanol, isopropanol, propylene glycol, glycerine, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerine, and, in particular, one or more thickeners, which may advantageously be selected from the group consisting of silicon dioxide, aluminium silicates, polysaccharides and derivatives thereof, for example hyaluronic acid, xanthan gum, hydroxy-propylmethylcellulose, particularly advantageously from the group consisting of the polyacrylates, preferably a polyacrylate from the group consisting of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

The aqueous phase may comprise hydrophilic surfactants. The hydrophilic surfactants are preferably selected from the group consisting of the alkylglucosides, acyl lactylates, betaines and coconut amphoacetates.

The aqueous phase my comprise hydrophilic thickeners. The hydrophilic thickeners are preferably selected from the following group: carboxyvinyl polymers, such as, for example, Carbopols (carbomers) from Noveon and Pemulen products (acrylate/C10-C30-alkyl acrylate copolymer); polyacrylamides, such as, for example, the crosslinked copolymer with the trade name Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) from SEPPIC; 2-acrylamido-2-methylpropanesulfonic acid polymer and copolymer, which may optionally be crosslinked or neutralised, such as, for example, poly(2-acrylamido-2-methylpropanesulfonic acid), marketed under the trade name “Hostacerin AMPS” (CTFA name: ammonium polyacryldimethyltauramide); cellulose-based derivatives, such as, for example, hydroxyethylcellulose; polysaccharides and in particular xanthan gum; and mixtures thereof.

Emulsifiers which are particularly preferably used for the preparation of W/O emulsions and creams are, inter alia, the following:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol mono-stearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Further advantageous W/O emulsifiers are sorbitan glycerol and/or sugar alkyl esters or ethers; silicone surfactants, such as, for example, a mixture of dimethicone copolyol, marketed under the trade name “DC 5225 C” by Dow Corning, and alkyldimethicone copolyols, such as, for example, the laurylmethicone copolyol marketed under the trade name “Dow Corning 5200 Formulation Aid” by Dow Corning; cetyldimethicone copolyol, such as, for example, the commercial product Abil EM 90R from Goldschmidt, a mixture of cetyldimethicone copolyol, consisting of polyglyceryl isostearate (4 mol) and hexyl laurate, marketed under the trade name Abil WE 09 by Goldschmidt.

One or more co-emulsifiers may additionally be used in combination with polyol alkyl esters, for example glycerol and/or sorbitan esters, for example polyglyceryl isostearate, which is commercially available under the name Isolan GI 34 from Goldschmidt; sorbitan isostearate, as marketed, for example, under the name Arlacel 987 by Uniqema (Croda); sorbitan glyceryl isostearate, marketed under the trade name Arlacel 986 by Uniqema (Croda), and mixtures thereof.

Examples of suitable emulsifiers for O/W emulsions are nonionic emulsifiers, such as, for example, ethoxylated (especially polyethoxylated) fatty acid esters of glycerine, ethoxylated sorbitan fatty acid esters; ethylenoxylated and/or propylenoxylated fatty acid esters of sugars, such as, for example, sucrose stearate; fatty alcohol ethers of sugar, such as, for example, polyalkylglucosides (APG), such as, for example, decylglucoside and laurylglucoside, as available, for example, under the trade name Plantaren from Cognis. Cetostearyl glucoside pure or as a mixture, such as, for example, in the commercial product Montanov 68 from Seppic; TegoCare CG 90 (Goldschmidt); Emulgade KE3302 (Cognis/Henkel), may also be present. Possible O/W emulsifiers are also formed by compounds of arachidyl glucoside, such as, for example, as a mixture with arachidyl alcohol, behenyl alcohol and arachidyl glucoside, marketed under the trade name Montanov 202 by SEPPIC.

Individual emulsifiers may also be mentioned below by way of example of the chemical classes of substance described, which can be employed in the described types of emulsions.

The following products are trademarks of Degussa Goldschmidt:

| | |
|---|---|
| Abil Care 85 | Dimethicone Copolyol (and) Caprylic/Capric |
| | Triglycerides |
| Abil EM 90 | Cetyl PEG/PPG-10/1 Dimethicone |
| Abil EM 97 | Bis-PEG/PPG-14/14 Dimethicone (and) Cyclopenta- |
| | siloxane |
| Abil WE 09 | Polyglyceryl-4 Isostearate (and) Cetyl Dimethicone |
| | Copolyol (and) Hexyl Laurate |
| Tego Care 150 | Glyceryl Stearate (and) Steareth-25 (and) Ceteth-20 |
| | (and) Stearyl Alcohol |
| Tego Care 215 | Ceteareth-15 (and) Glyceryl Stearate |
| Tego Care 450 | Polyglyceryl-3 Methylglucose Distearate |
| Tego Care CG 90 | Cetearyl Glucoside |
| Tego Care PS | Methyl Glucose Sesquistearate |
| TEGO Care 165 | Glyceryl Stearate (and) PEG-100 Stearate |
| ISOLAN GPS | Polyglyceryl-4 Diisostearate and |
| | Polyhydroxystearate Sebacate |
| TEGO Care CE 40 | Cetearyl Alcohol; Palmitamidopropyltrimonium |
| | Chloride |
| TEGO SIS 40 | PEG-40 Sorbitan Perisostearate |

The following products are trademarks of Cognis Deutschland:

| | |
|---|---|
| Emulgade F | Cetearyl Alcohol (and) PEG-40 Castor Oil (and) |
| | Sodium Sulfate |
| Emulgade 1000Ni | Cetearyl Alcohol (and) Ceteareth-20 |
| Emulgade CM | Cetearyl Isononanoate (and) Ceteareth-20 (and) |
| | Cetearyl Alcohol (and) Glyceryl Stearate (and) |
| | Glycerin (and) Ceteareth-12 (and) Cetyl Palmitate |
| Eumulgin VL 75 | Lauryl Glucoside (and) Polyglyceryl-2 Dipolyhydroxy- |
| | stearate (and) Glycerin |
| Emulgade sucro | Sucrose Polystearate (and) Hydrogenated Polyiso- |
| | butene |
| Eumulgin SG | Sodium Stearoyl Glutamate |
| Dehymuls HRE-7 | PEG-7 Hydrogenated Castor Oil |
| Dehymuls LE | PEG-30 Dipolyhydroxystearate |
| Dehymuls PGPH | Polyglyceryl-2 Dipolyhydroxystearate |

The following products are trademarks of Uniqema, Belgium

| | |
|---|---|
| ARLATONE 2121 | Sorbitan Stearate (and) Sucrose Cocoate |
| ARLATONE LC | Sorbitan Stearate (and) Sorbityl Laurate |
| | ARLATONE V-100 Steareth-100 (and) Steareth-2 |
| | (and) Glyceryl Stearate Citrate (and) Sucrose (and) |
| | Mannan (and) Xanthan Gum |
| ARLATONE V-175 | Sucrose Palmitate (and) Glyceryl Stearate (and) |
| | Glyceryl Stearate Citrate (and) Sucrose (and) |
| | Mannan (and) Xanthan Gum |
| ARLACEL 1689V | Sorbitan Oleate (and) Polyglyceryl-3 Polyricinoleate |
| ARLACEL 1690 | Sorbitan Isostearate (and) Polyglyceryl-3 Poly- |
| | ricinoleate |
| ARLACEL 186 | Glyceryl Oleate (and) Propylene Glycol |
| ARLACEL 481V | Sorbitan Oleate (and) Hydrogenated Castor Oil |
| | (and) Beeswax (and) Stearic Acid |
| ARLACEL 582 | Sorbitan Isostearate (and) PEG-2 Hydrogenated |
| | Castor Oil (and) Ozokerite (and) Hydrogenated |
| | Castor Oil Solid |
| ARLACEL 83V | Sorbitan Sesquioleate |
| ARLACEL 986 | Sorbitan Isostearate (and) Hydrogenated Castor Oil |
| | (and) Beeswax (and) Stearic Acid |
| ARLACEL 987 | Sorbitan Isostearate |
| ARLACEL 989 | PEG-7 Hydrogenated Castor Oil |
| ARLACEL P135 | PEG-30 Dipolyhydroxystearate |
| PRISORINE 3700 | Polyglyceryl-3 Diisostearate |
| PRISORINE 3791 | Polyglyceryl-2 Isostearate |
| SPAN 20 | Sorbitan Laurate |
| SPAN 80V | Sorbitan Oleate |
| SPAN 85V | Pharma Sorbitan Trioleate Liquid |

The following products are trademarks of Tri-K Ind.:

| | |
|---|---|
| Biobase EP | Glyceryl Stearate, Cetearyl Alcohol, Sodium Stearoyl |
| | Lactylate, Lecithin |
| Biobase RS | Glyceryl Stearate, Cetearyl Alcohol, Sodium Stearoyl |
| | Lactylate, Tocopherol |

The following products are trademarks of Vama FarmaCosmetica SrI

| | |
|---|---|
| Emulvama AGC | Glyceryl Stearate, Cetearyl Alcohol, Stearic Acid, |
| | Sodium Cocoyl Glutamate |
| Emulvama AGC | Glyceryl Stearate, Cetearyl Alcohol, Stearic Acid, |
| | Sodium Cocoyl Glutamate |
| Emulvama AGW | Sodium Cocoyl Glutamate, Sodium Cocoyl |
| | Hydrolyzed |
| | Wheat Protein, Disodium Capryloyl Glutamate, |
| | Potassium Cocoyl PCA |

The invention therefore relates further to the use of at least one kind of inorganic pigments composed of a spherical composite particle comprising at least one metal oxide to enhance the sun protection of a water-in-oil emulsion comprising at least one organic UV filter and/or at least one inorganic UV filter.

The invention therefore relates further to the use of at least one kind of inorganic pigments composed of a spherical composite particle comprising at least one metal oxide to enhance the sun protection of a water-in-silicone emulsion comprising at least one organic UV filter and/or at least one inorganic UV filter.

The invention therefore relates further to the use of at least one kind of inorganic pigments composed of a spherical composite particle comprising at least one metal oxide to enhance the sun protection of a gel comprising at least one organic UV filter and/or at least one inorganic UV filter.

The invention therefore relates further to the use of at least one kind of inorganic pigments composed of a spherical composite particle comprising at least one metal oxide to enhance the sun protection of an oil-in-water emulsion comprising at least one organic UV filter and/or at least one inorganic UV filter.

One kind of inorganic pigments composed of a spherical composite particle comprising at least one metal oxide differs from another kind of inorganic pigments composed of a spherical composite particle comprising at least one metal oxide through their particle size and/or their particle size distribution and/or their materials used as substrates and/or their materials used in their coatings and/or their number of coatings and/or their sequence of coatings.

There is no limitation to the kind of organic or inorganic UV filter present in the cosmetic formulations as described before or preferably described before. Besides the inorganic pigments, the cosmetic formulations may comprise at least one organic UV filter, so-called hydrophilic or lipophilic sun-protection filters, which are mostly effective in the UVA region and/or UVB region (absorbers). These substances can be selected, in particular, from dibenzoylmethane derivatives, cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO 93/04665. The said UV filters are usually named below in accordance with INCI nomenclature.

Particularly suitable organic UV filter as single organic UV filter or as part of a mixture of UV filters of the cosmetic formulation as described before or preferably described before are:

Dibenzoylmethane derivatives, in particular 4-isopropyldibenzoylmethane and 4,4′-methoxy-tert-butyldibenzoylmethane, which are described in the French patent applications FR-A-2 326 405 and FR-A-2 440 933 and in European patent application EP-A-0 114 607. 4,4′-Methoxy-tert-butyldibenzoylmethane is currently commercially available from Merck under the trade name Eusolex® 9020.

Para-aminobenzoic acid and derivatives thereof: PABA, Ethyl PABA, Ethyl dihydroxypropyl PABA, Ethylhexyl dimethyl PABA, for example marketed under the name “Escalol 507” by ISP, Glyceryl PABA, PEG-25 PABA, for example marketed under the name “Uvinul P25” by BASF.

Salicylates: Homosalate (3,3,5-trimethyl-cyclohexyl-salicylate) marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, etocrylene, for example marketed by BASF under the name “Uvinul N35”. Furthermore, for example, methoxycrylene, marketed by Hallstar under the name “Solastay S1”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl) benzoate or 2-hydroxy-4-methoxybenzophenone, marketed by Merck, Darmstadt, under the name “Eusolex® 4360”.

Benzylidenecamphor derivatives: 3-Benzylidenecamphor, for example marketed under the name “Mexoryl SD” by Chimex, 4-Methylbenzylidene-camphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidene-dicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzo-triazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: Ttris Biphenyl Triazine (TBPT) of BASF, Ethylhexyltriazone, for example marketed by BASF under the name “Uvinul T150”, Diethylhexylbutamidotriazone, for example marketed by Sigma 3V under the name “Uvasorb HEB”. Further triazine derivatives are by way of example 2,4,6-tris(diisobutyl 4′-aminobenzalmalonate)-s-triazine or 2,4,6-tris(biphenyI)-1,3,5-triazine, or butyl 4-({4-{[4-(butoxycarbonyl)phenyl]amino}-6-[(3-{1,3,3,3-tetramethyl-1-[(trimethylsilyl)oxy]disiloxanyl} propyl)amino]-1,3,5-triazin-2-yl}amino)benzoate, marketed under the name “Mexoryl SBS” and bis-ethylhexyloxyphenol methoxyphenyl triazine, for example marketed by BASF under the name “Tinosorb S”. Structure of Mexoryl SBS:

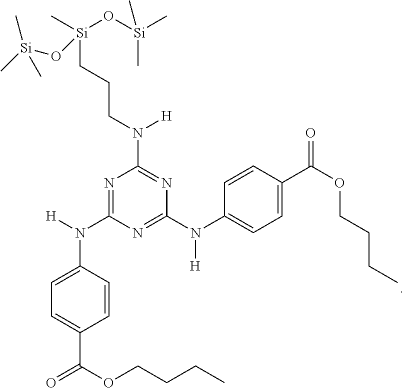 [SMILES: CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

Anthraniline derivatives: Menthyl anthranilate, for example marketed by Symrise under the name “Neo Heliopan MA”.

Imidazole derivatives: ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyI)-4,4-diphenylbutadiene.

Benzoxazole derivatives: 2,4-bis[5-(1-dimethylpropyl)benzoxazol-2-yl(4-phenyl) imino]-6-(2-ethylhexyl)imino-1,3,5-triazine, for example marketed by Sigma 3V under the name Uvasorb K2A, and mixtures comprising this.

Piperazine derivatives, such as, for example, the compound

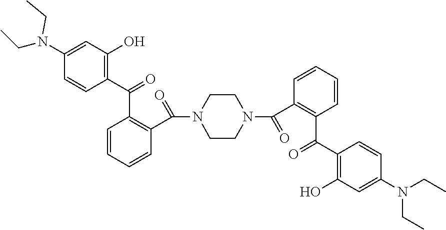 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

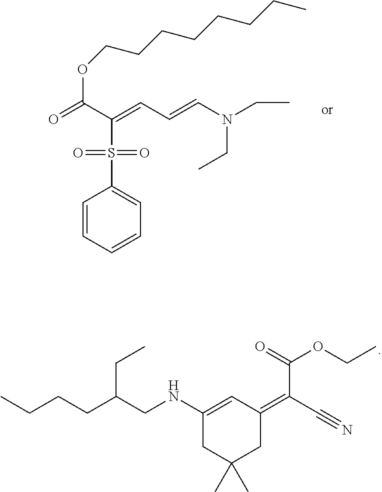

Preference is also given to UV filters based on polysiloxane copolymers having a random distribution in accordance with the following formula, where, for example, a=1.2; b=58 and c=2.8:

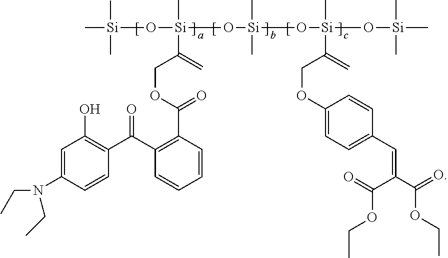

The compounds listed should only be regarded as examples. It is of course also possible to use other UV filters.

Suitable organic UV-protecting substances as part of the cosmetic formulation to be enhanced can preferably be selected from the following list: Homosalate, Octocrylene, Ethylhexyl salicylate, Butyl Methoxy-dibenzoylmethane, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidene-dicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-Bis[5-(1-dimethylpropyl)benzoxazol-2-yl(4-phenyl)-imino]-6-(2-ethylhexyl)imino-1,3,5-triazine and mixtures thereof.

Particular preferable organic UV-protecting substances as part of the cosmetic formulation to be enhanced are Homosalate, Octocrylene, Ethylhexyl salicylate and/or Butyl Methoxy-dibenzoylmethane.

These organic UV filters are generally incorporated into the cosmetic formulation in an amount of 0.01 per cent by weight to 20 per cent by weight, preferably 1% by weight-10% by weight.

Besides the inorganic pigments, the cosmetic formulations may comprise at least an inorganic UV filter, so-called particulate UV filters.

Preference is given here to those inorganic UV filters from the group of the titanium dioxides, such as, for example, coated titanium dioxide (for example Eusolex® T-2000, Eusolex®T-AQUA, Eusolex®T-AVO, Eusolex® T-PRO, Eusolex® T-EASY, zinc oxides (for example RonaCare® Zinc Oxide or Sachtotec®), iron oxides or also cerium oxides and/or zirconium oxides.

Furthermore, combinations with pigmentary titanium dioxide or zinc oxide are also possible, where the particle size of these pigments is greater than or equal to 200 nm, for example Hombitan® FG or Hombitan® FF-Pharma.

It may furthermore be preferred to comprise inorganic UV filters which have been aftertreated by conventional methods, as described, for example, in Cosmetics & Toiletries, February 1990, Vol. 105, pp. 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Inorganic UV filters which are preferably comprised in the cosmetic formulation comprising the at least one inorganic pigment as described before are:
untreated titanium dioxides, such as, for example, the products Micro-titanium Dioxide MT 500 B from Tayca; titanium dioxide P25 from Degussa,
Aftertreated micronised titanium dioxides with aluminium oxide and silicon dioxide aftertreatment, such as, for example, the product “Microtitanium Dioxide MT 100 SA” from Tayca; or the product “Tioveil Fin” from Uniqema,
Aftertreated micronised titanium dioxides with aluminium oxide and/or aluminium stearate/laurate aftertreatment, such as, for example, Microtitanium Dioxide MT 100 T from Tayca, Eusolex T-2000 from Merck,
Aftertreated micronised titanium dioxides with iron oxide and/or iron stearate aftertreatment, such as, for example, the product “Microtitanium Dioxide MT 100 F” from Tayca,
Aftertreated micronised titanium dioxides with silicon dioxide, aluminium oxide and silicone aftertreatment, such as, for example, the product “Micro-titanium Dioxide MT 100 SAS” from Tayca,
Aftertreated micronised titanium dioxides with sodium hexametaphosphates, such as, for example, the product “Microtitanium Dioxide MT 150 W” from Tayca.
Untreated zinc oxides, such as, for example, the product Z-Cote from BASF (Sunsmart), Nanox from Elementis
Aftertreated zinc oxides, such as, for example, the following products:
“Zinc Oxide CS-5” from Toshibi (ZnO aftertreated with polymethyl-hydrogenosiloxanes)
Nanogard Zinc Oxide FN from Nanophase Technologies
“SPD-Z1” from Shin-Etsu (ZnO aftertreated with a silicone-grafted acrylic polymer, dispersed in cyclodimethylsiloxanes)
“Escalol Z100” from ISP (aluminium oxide-aftertreated ZnO dispersed in an ethylhexyl methoxycinnamate/PVP-hexadecene/methicone copolymer mixture)
“Fuji ZNO-SMS-10” from Fuji Pigment (ZnO aftertreated with silicon dioxide and polymethylsilsesquioxane)
Untreated cerium oxide micropigment, for example with the name “Colloidal Cerium Oxide” from Rhone Poulenc;
Untreated and/or aftertreated iron oxides with the name Nanogar from Arnaud.

The treated micronised titanium dioxides may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3 from Cardre,
polydimethylhydrogenosiloxanes, such as, for example, the product Microtitanium Dioxide USP Grade Hydrophobic from Color Techniques.

For example, it is also possible to employ mixtures of various inorganic UV filters, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide, zinc oxide mixtures, such as, for example, the product UV-Titan M261 from Sachtleben, in the cosmetic formulation as described before.

These inorganic UV filters are generally incorporated into the cosmetic formulation in an amount of 0.1% by weight to 25% by weight, preferably 2% by weight to 10% by weight.

Preferably, the cosmetic formulation comprises organic and inorganic UV filters as described or preferably described before.

The invention therefore relates to the use as described before or preferably described before or below wherein the cosmetic formulation comprises organic and inorganic UV filters.

Particularly preferably, the cosmetic formulation comprises only organic UV filters as described or preferably described before.

The invention therefore relates to the use as described before or preferably described before or below wherein the cosmetic formulation comprises only organic UV filters.

Preferred inorganic pigments to be used according to the invention are composed of spherical composite particles comprising at least one metal oxide wherein the metal oxide is independently of each other selected from the group zirconium oxide, titanium dioxide, zinc oxide, cerium oxide or mixtures thereof.

Particular preferred inorganic pigments to be used according to the invention are composed of spherical composite particles comprising at least one metal oxide wherein the metal oxide is independently of each other selected from titanium dioxide or a mixture of titanium dioxide with zirconium oxide, zinc oxide or cerium oxide.

The invention therefore relates further to the use as described before or preferably described before wherein the metal oxide of the kinds of inorganic pigments composed of a spherical composite particle comprising at least one metal oxide is independently of each other selected from the group zirconium oxide, titanium dioxide, zinc oxide, cerium oxide or mixtures thereof.

Particularly preferred inorganic pigments to be used according to the invention are composed of spherical composite particles comprising at least one metal oxide wherein the metal oxide or the mixture of metal oxides is or are coated with agglomerates on the surface of the spherical base particle.

The invention therefore relates further to the use as described before or preferably described before wherein the metal oxide or the mixture of metal oxides is/are coated with agglomerates on the surface of the spherical base particle of the inorganic pigments according to the invention.

Agglomerates in this application are taken to mean oxide particles of the metal oxide(s) distributed irregularly on the surface of the spherical base particles. The formation of agglomerates reduces the number of light-scattering centres, and the scattering capacity of the coating increases less with increasing coverage than in the case of coating with non-aggregated individual particles. The partial vertical arrangement of a plurality of oxide particles to give agglomerates also results in an irregular layer thickness of the coating and in a fissured surface. The interaction with the medium is therefore more intense, and networks are able to form between the coated spherical particles, causing a desired increase in viscosity and at the same time improving the application behaviour.

Coating(s) in this patent application are taken to mean the partial or complete covering of the spherical base particles with metal oxide(s) in the form of agglomerates.

The spherical base particles are coated on the surface (1st layer) with agglomerates, which comprise a metal oxide or a mixture of metal oxides as described before or preferably described before.

The spherical base particles preferably have a coating of agglomerates comprising TiO₂. The TiO₂ here can be in the rutile or anatase modification, preferably in the anatase form.

It is therefore preferred that the inorganic pigment composed of a spherical composite particle comprising at least one metal oxide is a spherical base particle preferably having a coating of agglomerates comprising TiO₂ as described before.

Since the agglomerates are distributed very irregularly on the surface of the spherical base particle, the average layer thickness of the agglomerates, i.e. the layer thickness for an assumed uniform and compact (non-agglomerated) distribution of the layer material on the substrate surface, is indicated in this application.

The average layer thickness of the first layer comprising the at least metal oxide as described before or preferably described before is preferably 0.01-2 μm, in particular 0.02-1 μm, very particularly preferably 0.05-0.8 μm.

Preferred inorganic pigments to be used according to the invention are composed of spherical composite particles comprising at least one metal oxide wherein the spherical base particle is independently of each other selected from aluminium silicate, alkali metal silicates, alkaline-earth metal silicates, alkali-metal aluminium silicates, alkaline-earth metal aluminium silicates, combinations of these silicates, silicon dioxide, glass spheres, hollow glass spheres, aluminium oxide or polymers.

Suitable alkaline-earth metal silicates are magnesium silicate and calcium silicate.

Suitable alkali metal aluminium silicates are sodium potassium aluminium silicate.

The invention therefore relates further to the use as described before or preferably described before wherein the spherical base particle of the kinds of inorganic pigments composed of the spherical composite particle is independently selected from magnesium silicate, aluminium silicate, alkali-metal aluminium silicates, alkaline-earth metal aluminium silicates, combinations of these silicates, silicon dioxide, glass spheres, hollow glass spheres, aluminium oxide or polymers.

Suitable polymers building the spherical base particles are ethylene/acrylic acid copolymers, ethylene/methacrylate copolymers, hexamethylene diisocyanate (HDI) based polymers, HDI/trimethylol hexyllactone copolymers, nylon, polyacrylates, polymethyl methacrylate copolymers, polyethylene, polystyrene, polymethylsilylsesquioxane and combinations thereof.

Suitable spherical base particles, for example, are commercially available, inter alia from Sunjin Chemical, Omega Materials, 3M, Dow Corning or Evonik. Preferred spherical base particles are selected from the group magnesium silicate, aluminium silicate, sodium potassium aluminium silicates or silicon dioxide.

Particularly preferred spherical base particles are magnesium silicates and/or sodium potassium aluminium silicates, as marketed, for example, by 3M under the trade names Ceramic Microspheres and Cosmetic Microspheres or Zeeospheres.

The spherical base particles have a particle diameter of 0.1-100 μm, preferably 0.3-50 μm, in particular 0.5-15 μm.

The inorganic pigments composed of spherical composite particles consisting of a spherical base particle as described or preferably described before which are coated on the surface with agglomerates comprising at least one metal oxide as described before or preferably described before may optionally be coated with a second layer (2nd layer) or with further layers comprising a further metal oxide or metal-oxide mixture, metal hydroxide, BaSO₄, a lake or Berlin Blue.

The second coating or further coating may be in form of agglomerates or a uniform and compact layer on the surface of the spherical base particle comprising the first coating of agglomerates.

Suitable metal oxides for the second coating or further coating may be selected from the group SiO₂, Al₂O₃, Fe₂O₃, Fe₃O₄, FeOOH or mixtures thereof. The agglomerate may furthermore also consist of BaSO₄ or a mixture of BaSO₄ and one or more of the said metal oxides. For the second coating Fe₂O₃ or Fe₃O₄ are preferred metal oxides.

A second coating or further coating may comprise a lake or Berlin Blue.

Lakes are organic colorants, which are bonded to an inorganic support matrix. Suitable as support matrix are, in particular, network-forming inorganic oxides, such as, for example, Al₂O₃ and SiO₂. Organic colorants which may be mentioned are all coloured compounds which can be converted into lakes, in particular the alkali and alkaline-earth metal salts of Carmine Red (CAS No. 1390-65-4), Allura Red (CAS No. 25956-17-6), tartrazine (CAS No. 1934-21-0), Brilliant Blue FCF (CAS No. 3844-45-9), erythrosine (CAS No. 16423-68-0) and Phloxine B (CAS No. 18472-87-2). This list only represents a small number of the possible lakes and should not be understood as being restrictive.

Berlin Blue is the iron(III) salt of the hexacyanoferrate(II) anion, having the empirical formula Fe₄[Fe(CN)₆]₃. It can easily be prepared by combining the solutions of potassium hexacyanoferrate(II) and iron(III) salts or potassium hexacyanoferrate(III) and iron(II) salts in weakly acidic solution.

The average layer thicknesses of the second layer or each further coating comprising metal oxide, lake or Berlin Blue are 10-500 nm, preferably 10-200 nm and particularly preferably 10-100 nm. The proportion by weight of the lake in the pigment as a whole is dependent on the desired colour intensity of the product and the colouring effect of the respective lake. It is 0.1-30%, preferably 0.2-25% and particularly preferably 0.5-20%.

In order to improve the dispersibility, the chemical and photochemical stability and the skin feel, it is frequently advisable to apply a top layer (final layer) comprising SiO₂. The SiO₂ layer generally has an average layer thickness of 0.01-1 μm, in particular 0.02-0.7 μm and very particularly preferably 0.05-0.5 μm.

This final SiO₂ layer sheathes the agglomerate-coated spherical particles in a thin layer. It improves the dispersibility and prevents chemical and photo-chemical interactions of the spherical composite particle to be used according to the invention with other ingredients of the cosmetic formulation.

The inorganic pigments to be used according to the invention are preferably spherical composite particles consisting of a spherical base particle coated with one or two layers, the first layer comprising the metal oxide(s), as described before for the first layer and a final layer of SiO₂ as described before. Particular preference is given to spherical composite particles consisting of a spherical particle of alkali metal aluminium silicate coated with TiO₂ as described before and a final layer of SiO₂ as described before.

The spherical composite particles as described before or preferably described before have a particle diameter of 0.1-100 μm, preferably 0.1-60 μm, particularly preferably 0.1-25 μm.

In a preferred embodiment of the invention, one single kind of inorganic pigments composed of a spherical composite particle comprising at least one metal oxide enhances the sun protection of cosmetic formulations comprising at least one organic UV filter and/or at least one inorganic UV filter, preferably the sun protection of a water-in-oil emulsion, a water-in-silicone emulsion, a gel or a oil-in-water emulsion comprising at least one organic UV filter and/or at least one inorganic UV filter.

The invention therefore relates to the use as described before wherein one kind of inorganic pigments composed of spherical composite particles comprising at least one metal oxide enhances the sun protection of the cosmetic formulation.

The one single kind of inorganic pigments to be used according to the invention is particularly preferably the kind of inorganic pigments commercially available under the trade name RonaFlair® LDP White of Merck.

In an alternative embodiment of the invention, two kinds of inorganic pigments composed of a spherical composite particle comprising at least one metal oxide may be used according to the invention.

In an alternative preferred embodiment of the invention, two kinds of inorganic pigments composed of spherical composite particles comprising at least one metal oxide enhance the sun protection of cosmetic formulations comprising at least one organic UV filter and/or at least one inorganic UV filter, preferably the sun protection of a water-in-oil emulsion, a water-in-silicone emulsion, a gel or an oil-in-water emulsion comprising at least one organic UV filter and/or at least one inorganic UV filter.

The invention therefore relates to the use as described before or preferably described before wherein two kinds of inorganic pigments composed of spherical composite particles comprising at least one metal oxide enhance the sun protection of the cosmetic formulation.

In case two kinds of inorganic pigments composed of spherical composite particles comprising at least one metal oxide are present, the amount of the second kind of inorganic pigment is between 2 to 50% by weight related to the total amount of the inorganic pigments composed of a spherical composite particle comprising at least one metal oxide.

In case two kinds of inorganic pigments composed of spherical composite particles comprising at least one metal oxide are present, the amount of the second kind of inorganic pigment is preferably between 1 to 5% by weight related to the total amount of the inorganic pigments composed of spherical composite particles comprising at least one metal oxide.

The invention therefore relates to the use as described before or preferably described before wherein the amount of the second kind of inorganic pigment composed of spherical composite particles comprising at least one metal oxide is between 2 to 50% by weight related to the total amount of the inorganic pigments.

Within this embodiment of the invention, both kinds of inorganic pigments composed of a spherical composite particle preferably have the same spherical base particles but different coatings. Preferably, the spherical base particles of the two kinds of inorganic pigments are SiO₂ spheres.

The invention therefore relates to the use as described before or preferably described before wherein the spherical base particles of the two kinds of inorganic pigments are the same.

In a preferred embodiment, the first kind of inorganic pigment is composed of a spherical composite particle consisting of a SiO₂ sphere with agglomerates of a metal oxide described for the first layer, preferably TiO₂, and the second kind of inorganic pigment is composed of a spherical composite particle consisting of a SiO₂ sphere with agglomerates of the metal oxide described useful for the first layer, preferably TiO₂, and the second coating of metal oxides useful for the second layer, preferably iron oxides (Fe₂O₃/Fe₃O₄). Both kinds of inorganic pigments may optionally have a top layer of SiO₂. The amount of the second kind of inorganic pigment is between 2 to 50 weight % related to the total amount of the inorganic pigments. Preferably, the more colourless kind of inorganic pigment is seen as the first kind of inorganic pigments and the more coloured inorganic pigments are seen as the second kind of inorganic pigments thus setting the mass tone of the pigment mixture.

The inorganic pigment mixture as described before to be used according to the invention is preferably the inorganic pigment mixture commercially available under the trade name RonaFlair® Flawless of Merck.

The use level of the total of inorganic pigments used according to the invention in the cosmetic formulation which shall be enhanced is between 0.1 to 10% by weight, preferably by 2 to 4% by weight.

The preparation of the inorganic pigments as described herein is known, e.g. from EP 2316891 A1. Thus, for example, Mg or Al silicate spheres are generally melted by heating the finely divided silicate raw materials and/or oxidic starting compounds thereof in a gas stream and thus adopt a spherical shape. In order to make the melting easier by lowering the melting point, finely divided alkali and/or alkaline-earth metal compounds are typically added to the reaction mixture.

The coating of the spherical base particles can be carried out in a one-pot process. The spherical base particles can be coated with one or more coatings by wet-chemical coating or by the CVD or PVD process.

The coating of the spherical base particles is preferably carried out in the wet-chemical method by hydrolytic deposition or the metal oxides or metal hydroxides from salt solutions thereof. The formation of agglomerates can be caused by a suitable choice of the precipitation conditions. These are, in particular, the reaction temperature, the pH, the stirring speed and the metering rate of the salt solutions. TiO₂ precipitations are typically carried out in the pH range from 1.0 to 3.0. The tendency towards agglomerate formation increases from pH 1.0 to pH 3.0. However, if an excessively high pH is selected, so-called secondary precipitation may occur, i.e. the TiO₂ particles precipitate alongside the spherical base particles and do not form a layer. For the said base particles, different precipitation conditions, which can easily be determined by the person skilled in the art in the area of pigments, should generally be selected owing to the different surface behaviour.

The cosmetic formulations as described before or preferably described before can be prepared by processes, which are well known to the person skilled in the art, in particular by the processes, which serve for the preparation of water-in-oil emulsions, water-in-silicone-emulsions, gels and oil-in-water emulsions.

The inorganic pigments as described before or preferably described before can be incorporated even after the emulsion/gel is already prepared.

The following examples describe special cosmetic formulations. Generally, a basis formulation without an inorganic pigment is prepared to document the sun protection of the cosmetic formulation as such through the existing UV filters.

Different inorganic pigments are then incorporated into the basis formulation and the sun protection factor is measured. The results can be directly compared and show that inorganic pigments composed of a spherical composite particles comprising an inorganic UV filter are advantageous over uncoated silica spheres or the platelet based composite particles such as NS Boost.

In addition, a cosmetic formulation is prepared without any organic or inorganic UV filter but comprising at least one special kind of inorganic pigments composed of a spherical composite particle comprising at least one metal oxide. This formulation shows no UV absorption which document that the small amount of titanium dioxide on the surface of the spherical base particle does not contribute to the observed technical effect.

The below data clearly show that the use of inorganic pigments composed of a spherical composite particle comprising at least one metal oxide as described before or preferably described before surprisingly enhance the sun protection of a cosmetic formulation as described before or preferably described before comprising at least an organic UV filter and/or at least an inorganic UV filter by 10 to 40%.

The following examples explain the present invention in greater detail without restricting the scope of protection. In particular, the features, properties and advantages described in the examples of the inorganic pigments used in the cosmetic formulations can also be applied to other inorganic pigments, which are not mentioned in detail, but fall within the scope of description, unless stated otherwise elsewhere. In addition, the invention can be carried out throughout the range claimed and is not restricted to the examples mentioned here.

### EXAMPLES

The inorganic pigments and ingredients used within the cosmetic formulations are commercially available and described with the internationally accepted INCI nomenclature.

The inorganic pigments used in the examples are described with the term “filler” in the formulation examples.

### Example 1

A water-in-oil emulsion is prepared comprising four organic UV filters.

The following table 1 shows the composition of the water-in-oil emulsion with organic UV-filters. The amounts are given in weight %.

TABLE 1

| Phase | Ingredient [INCI] | [%] |
|---|---|---|
| A | Homosalate | 10.00 |
| | Octocrylene | 10.00 |
| | Ethylhexyl Salicylate | 5.00 |
| | Butyl | 5.00 |
| | Methoxydibenzoylmethane | |
| | PEG-30 | 2.50 |
| | Dipolyhydroxystearate | |
| | Dicaprylyl Ether | 10.00 |
| | Dicaprylyl Carbonate | 2.00 |
| | Bis-Ethylhexyl | 1.00 |
| | Hydroxydimethoxy | |
| | Benzylmalonate | |
| | C10-30 Alkyl Acrylate | 0.50 |
| B | Aqua | add |
| | | to 00 |
| | p-Anisic Acid | 0.15 |
| | Sodium Chloride | 0.50 |
| | PEG-8 Laurate | 0.20 |
| | Poloxamer 407 | 0.50 |
| | Benzyl Alcohol | 0.10 |
| | Benzyl Salicylate | 0.10 |
| | Disodium EDTA | 0.05 |
| | Caprylyl Glycol | 0.50 |
| | Styrene/Acrylates | 3.00 |
| | Copolymer | |
| C | Dimethicone | 5.00 |
| | Cyclopentasiloxane | 2.00 |
| | Isododecane, | 2.00 |
| | Disteardimonium Hectorite, | |
| | Propylene Carbonate | |
| | Lauryl PEG/PPG-18/18 | 0.80 |
| | Methicone | |
| | Polymethylsilsesqioxane | 1.00 |
| D | Phenoxyethanol, | 0.50 |
| | Ethylhexyl Glycerin | |
| E | filler | 3.00 |

TABLE 2
used fillers are:

| sample | Composition of filler | Name |
|---|---|---|
| 1 | without filler | Basis |
| | | formulation |
| 2 | Sodium Potassium | RonaFlair ® LDP |
| | Aluminium Silicate, | White |
| | CI 77891, Silica | |
| 3 | Silica, CI 77891, CI | RonaFlair ® |
| | 77491 | Flawless |
| 4 | Mica, Silica | NS Boost |
| 5 | Silica | uncoated silica |
| | | spheres |

Emulsion Preparation:

Mix phase A and B separately and heat up to 80° C. After reaching the temperature, add Phase B to A while stirring. Add the premixed phase C and homogenize afterwards with e.g. Ultra-Turrax T-50 at 5000 rpm for 1.5 minutes. After homogenization let the emulsion cool down to room temperature under continuous stirring and add phase D and E one after another under stirring until everything is well dispersed.

The in vitro SPF determination was carried out one week after the preparation of the emulsions.

Measurement:

To determine the sun protection factor 32.5±0.5 mg of the emulsion was applied on a UV-permeable PMMA sheet with unilaterally defined roughening and an area of 25 cm² distributed (HD Heliplate of the company Helio Screen Labs Laboratories). The samples were dried for 20 minutes in the dark in order to achieve the formation of a stabilized product film. The transmittance of the formulation was detected with the UV-spectrophotometer UV-2000S (Transmissionsanalysator Labsphere). The measurements were performed on 5 measuring points of the plate. The calculation of the in vitro SPF was done with the built-in software of the spectrophotometer.

Three plates for each samples were prepared. This yields for each emulsion 15 measuring points. The measurement results of the SPF determination are listed in the following table:

TABLE 3
results in vitro SPF:

| sample | Composition of filler | in vitro SPF |
|---|---|---|
| 1 | without filler | 30.0 |
| 2 | Sodium Potassium Aluminium | 65.6 |
| | Silicate, CI 77891, Silica | |
| 3 | Silica, CI 77891, CI 77491 | 64.6 |
| 4 | Mica, Silica | 28.5 |
| 5 | Silica | 32.4 |

Measurement In Vivo According to ISO 24444, 2010:

To determine the sun protection factor 2.0 mg/cm²±2.5% of the formulations (samples 1, 2, 3 and 6 as explained before or below) were applied quickly in small droplets with a syringe all over the test area (back of the volunteers), and spread by gently rubbing (soft touch) with a non-saturated finger-cot, first with rotating followed by crosswise movements for a final uniform coating on the skin. The spreading time was between 20 and 50 seconds.

After colorimetric estimation of the skin type through the measurement of ITA° with chromameter (CR 300, Minolta, Langenhagen) an untreated area was irradiated first to detect the unprotected MED. The increment was 1.25.

Untreated and product treated areas were irradiated with a sun simulator with increasing amounts of UV-radiation. Between 15 and 30 minutes after product application the irradiation of the product treated test areas started. On each test area 6 small circular spots of approximately 1 cm² were irradiated with increasing doses. The increment was 1.12. The detected MED dose was irradiated on the fourth step of the six doses of irradiation.

The sun simulator units used for the study (300 W Multiport, SOLAR Light, Philadelphia, Pa., USA) complied with the ISO 24444, 2010. Irradiation times were estimated by skin color typing. The test areas were examined between 16 and 24 hours after irradiation, and the irradiation spot with the Minimal Erythemal Dose (MED) was determined for each treatment. By dividing the MED of the product treated test field by the MED of the untreated test field the individual sun protection factor was calculated.

Visual rating of skin reaction by a blind observer was performed 16 to 24 hours after irradiation. Scores were recorded on a score sheet and subsequently entered into a PC system with an appropriate computer program.

TABLE 4
results in vivo SPF:

| | | in vivo SPF | in vivo SPF |
|---|---|---|---|
| sample | Composition of filler | 6 volunteers | 10 volunteers |
| 1 | without filler | 28.7 | — |
| 2 | Sodium Potassium | 37.6 | 40.9 |
| | Aluminium Silicate, | | |
| | CI 77891, Silica | | |
| 3 | Silica, CI 77891, | 33.8 | 37.5 |
| | CI 77491 | | |

The following water-in-oil emulsion without any UV-filter but with the coated spherical fillers show no UV absorption resulting in an SPF of 1.7 measured in vivo as explained before.

The following table 5 shows the composition of the water-in-oil emulsion without UV-filters.

TABLE 5

| Phase | Ingredient [INCI] | [%] |
|---|---|---|
| A | PEG-30 Dipolyhydroxystearate | 2.50 |
| | Dicaprylyl Ether | 34.00 |
| | Dicaprylyl Carbonate | 8.00 |
| | Bis-Ethylhexyl Hydroxydimethoxy | 1.00 |
| | Benzylmalonate | |
| | C10-30 Alkyl Acrylate | 2.50 |
| B | Aqua | add to 100 |
| | p-Anisic Acid | 0.15 |
| | Sodium Chloride | 0.50 |
| | PEG-8 Laurate | 0.20 |
| | Poloxamer 407 | 0.50 |
| | Benzyl Alcohol | 0.10 |
| | Benzyl Salicylate | 0.10 |
| | Disodium EDTA | 0.05 |
| | Caprylyl Glycol | 0.50 |
| | Styrene/Acrylates Copolymer | 3.00 |
| C | Dimethicone | 5.00 |
| | Cyclopentasiloxane | 2.00 |
| | Isododecane, Disteardimonium | 2.00 |
| | Hectorite, Propylene Carbonate | |
| | Lauryl PEG/PPG-18/18 Methicone | 0.80 |
| | Polymethylsilsesqioxane | 1.00 |
| D | Phenoxyethanol, Ethylhexyl | 0.50 |
| | Glycerin | |
| E | filler | 3.00 |

TABLE 6
results in vivo SPF:

| | | in vivo SPF | in vivo SPF |
|---|---|---|---|
| Sample | Composition of filler | 6 volunteers | 10 volunteers |
| 6 | Sodium Potassium | 1.7 | — |
| | Aluminium Silicate, CI | | |
| | 77891, Silica | | |

### Example 2

A water-in-silicone emulsion is prepared comprising four organic UV filters.

The following table 7 shows the composition of the water-in-silicone emulsion with organic UV-filters. The amounts are given in weight %.

TABLE 7

| Phase | Ingredient [INCI] | [%] |
|---|---|---|
| A | Octocrylene | 10.00 |
| | Ethylhexyl Salicylate | 5.00 |
| | Homosalate | 10.00 |
| | Butyl Methoxydibenzoylmethane | 5.00 |
| | Bis-Ethylhexyl Hydroxydimethoxy | 1.00 |
| | Benzylmalonate | |
| | Lauryl PEG/PPG-18/18 Methicone | 4.00 |
| | Cyclopentasiloxane, Dimethicone | 5.00 |
| | Crosspolymer | |
| | Cyclopentasiloxane, Cyclohexasiloxane | 1.00 |
| | PPG-3 Myristyl Ether | 1.00 |
| B | Disodium EDTA | 0.10 |
| | Sodium Chloride | 2.50 |
| | Aqua | add to 100 |
| C | Propylene Glycol, Diazolidinyl Urea, | 0.50 |
| | Methylparaben, Propylparaben | |
| D | filler | 3.00 |

TABLE 8
used fillers are:

| sample | Composition of filler | Name |
|---|---|---|
| 1 | without filler | Basis formulation |
| 2 | Sodium Potassium Aluminium | RonaFlair ® LDP White |
| | Silicate, CI 77891, Silica | |
| 3 | Silica, CI 77891, CI 77491 | RonaFlair ® Flawless |
| 4 | Mica, Silica | NS Boost |
| 5 | Silica | uncoated silica spheres |

Emulsion Preparation:

Mix phase A and B separately and heat up to 60° C. After reaching the temperature, add phase B slowly to phase A while stirring. Homogenize afterwards with blade agitator at 2000 rpm for 2 minutes. Add phase C and homogenize with blade agitator at 2000 rpm for 2 minutes. Add phase D under stirring and homogenize with blade agitator at 1000 rpm for 2 minutes. The in vitro SPF determination was carried out one week after the preparation of the emulsions. The measurements were done as described in Example 1.

TABLE 9
results in vitro SPF:

| sample | Composition of filler | in vitro SPF |
|---|---|---|
| 1 | without filler | 8.8 |
| 2 | Sodium Potassium Aluminium | 15.0 |
| | Silicate, CI 77891, Silica | |
| 3 | Silica, CI 77891, CI 77491 | 17.6 |
| 4 | Mica, Silica | 9.2 |
| 5 | Silica | 12.0 |

### Example 3

A gel is prepared comprising two encapsulated organic UV filters. The encapsulated organic UV filters are marketed under the trade name Eusolex® UV Pearls® OB-S of Merck.

The following table 10 shows the composition of the gel with two encapsulated organic UV-filters. The amounts are given in weight %.

TABLE 10

| Phase | Ingredient [INCI] | [%] |
|---|---|---|
| A | Aqua (Water), Octocrylene, Butyl | 11.00 |
| | Methoxydibenzoylmethane, Silica, | |
| | PVP, Phenoxyethanol, Disodium | |
| | EDTA | |
| | Butylene Glycol | 4.00 |
| | Aqua | add to 100 |
| B | Sodium Acrylate, Acryloyl | 1.50 |
| | Dimethyltaurate, | |
| | Dimethylacrylamide Crosspolymer | |
| | Glycine Soja | 2.50 |
| | C12-15 Alkyl Benzoate | 2.50 |
| C | Phenoxyethanol, Ethylhexylglycerin | 1.00 |
| D | filler | 3.00 |

TABLE 11
used fillers are:

| sample | Composition of filler | Name |
|---|---|---|
| 1 | without filler | Basis formulation |
| 2 | Sodium Potassium Aluminium | RonaFlair ® LDP White |
| | Silicate, CI 77891, Silica | |
| 3 | Mica, Silica | NS Boost |
| 4 | Silica | uncoated silica spheres |

Emulsion Preparation:

Add phase A to phase B under stirring and homogenize afterwards with e.g. Ultra-Turrax T-50 at 5000 rpm for 1.5 minutes. Add phase C and D one after another under stirring until everything is well dispersed.

The in vitro SPF determination was carried out one week after the preparation of the emulsions. The measurements were done as described in Example 1.

TABLE 12
results in vitro SPF:

| sample | Composition of filler | in vitro SPF |
|---|---|---|
| 1 | without filler | 4.1 |
| 2 | Sodium Potassium Aluminium | 6.5 |
| | Silicate, CI 77891, Silica | |
| 3 | Mica, Silica | 5.0 |
| 4 | Silica | 5.3 |

### Example 4

An oil-in-water emulsion is prepared comprising an inorganic UV filter marketed under the trade name Eusolex®T-2000

The following table 13 shows the composition of the oil-in-water emulsion with organic UV-filters. The amounts are given in weight %.

TABLE 13

| Phase | Ingredient [INCI] | [%] |
|---|---|---|
| A | dipropylene glycol dibenzoate, ppg-15 stearyl | 6.00 |
| | ether benzoate, c12-15 alkyl benzoate | |
| | isohexadecane | 6.00 |
| | bis-hydroxyethoxypropyl dimethcione | 1.50 |
| | titanium dioxide, alumina, simethicone | 3.00 |
| | potassium cetyl phosphate | 2.50 |
| | steareth-2 | 0.30 |
| | steareth-21 | 0.60 |
| | cetearyl alcohol | 2.00 |
| | filler | 2.00 |
| B | aqua | add to 100 |
| | magnesium aluminium silicate | 0.20 |
| | Butylene glycol | 3.00 |
| | titanium dioxide, alumina, simethicone | 3.00 |
| C | Butylene glycol | 2.00 |
| | xanthan gum | 0.3 |
| | ammonium acryloyldimetyltaurate/vp copolymer | 0.2 |
| D | aqua, hyaluronic acid | 3.00 |
| | phenoxyethanol, methylparaben, ethylparaben, | 0.7 |
| | propylparaben | |

TABLE 14
used fillers are:

| sample | Composition of filler | Name |
|---|---|---|
| 1 | without filler | Basis formulation |
| 2 | Sodium Potassium Aluminium | RonaFlair ® LDP White |
| | Silicate, CI 77891, Silica | |

Emulsion Preparation:

Add phase C to phase B under stirring and heat up to 80° C. Mix phase A and heat up to 80° C. Add phase A to B/C under continuous stirring. Homogenize afterwards with e.g. Ultra-Turrax T-50 at 5000 rpm for 2 minutes and cool down to room temperature under continuous stirring. Add phase D under stirring until everything is well dispersed.

The in vitro SPF determination was carried out one week after the preparation of the emulsions. The measurements were done as described in Example 1.

TABLE 15
Results in vitro SPF:

| sample | Composition of filler | in vitro SPF |
|---|---|---|
| 1 | without filler | 12.6 |
| 2 | Sodium Potassium Aluminium | 14.7 |
| | Silicate, CI 77891, Silica | |

### Example 5

An oil-in-water emulsion is prepared comprising an inorganic UV filter marketed under the trade name Eusolex® T-AVO and two organic UV filter.

The following table 16 shows the composition of the oil-in-water emulsion with organic UV-filters. The amounts are given in weight %.

TABLE 16

| Phase | Ingredient [INCI] | [%] |
|---|---|---|
| A | Octocrylene | 10.00 |
| | Butyl Methoxydibenzoylmethane | 3.00 |
| | C12-15 Alkyl Benzoate | 9.90 |
| | Cetearyl Alcohol | 2.00 |
| | C14-22 Alcohols, C12-20 Alkyl Glucoside | 3.00 |
| | Titan Dioxide, Silica | 2.00 |
| | Propylparaben | 0.05 |
| | VP Hexadecene, Copolymer | 0.50 |
| B | Aqua | add to |
| | | 100 |
| | Glycerin | 2.00 |
| | Disodium EDTA | 0.10 |
| | Methylparaben | 0.15 |
| C | Magnesium Aluminium Silicate | 0.40 |
| | Xanthan Gum | 0.10 |
| D | Filler | 3.00 |

TABLE 17
used fillers are:

| sample | Composition of filler | Name |
|---|---|---|
| 1 | without filler | Basis formulation |
| 2 | Sodium Potassium Aluminium | RonaFlair ® LDP White |
| | Silicate, CI 77891, Silica | |
| 3 | Silica, CI 77891, CI 77491 | RonaFlair ® Flawless |

Emulsion Preparation:

Add phase C to phase B under stirring. Heat up phase A and B/C to 75° C. and add phase A to B/C. Homogenize afterwards with e.g. Ultra-Turrax T-50 at 5000 rpm for 2 minutes. Cool down under continuous stirring to room temperature and add phase D under while stirring until everything is well dispersed.

The in vitro SPF determination was carried out one week after the preparation of the emulsions. The measurements were done as described in Example 1.

TABLE 18
results in vitro SPF:

| sample | Composition of filler | in vitro SPF |
|---|---|---|
| 1 | without filler | 26.5 |
| 2 | Sodium Potassium Aluminium | 32.1 |
| | Silicate, CI 77891, Silica | |
| 3 | Silica, CI 77891, CI 77491 | 28.0 |

1. A method of enhancing the sun protection of a cosmetic formulation comprising at least one organic UV fileter and/or at least one inorganic UV filter, said method comprising adding to said formulation at least one kind of inorganic pigments composed of a spherical composite particle comprising at least one metal oxide.

2. The method according to claim 1 wherein the cosmetic formulation is a water-in-oil emulsion.

3. The method according to claim 1 wherein the cosmetic formulation is a water-in-silicone emulsion.

4. The method according to claim 1 wherein the cosmetic formulation is a gel.

5. The method according to claim 1 wherein the cosmetic formulation is an oil-in-water emulsion.

6. The method according to claim 1 wherein one kind of inorganic pigments composed of a spherical composite particle comprising at least one metal oxide enhances the sun protection of the cosmetic formulation.

7. The method according to claim 1 wherein two kinds of inorganic pigments composed of spherical composite particles comprising at least one metal oxide enhance the sun protection of the cosmetic formulation.

8. The method according to claim 1 wherein the amount of the second kind of inorganic pigment is between 2 to 50 weight % related to the total amount of the inorganic pigments.

9. The method according to claim 1 wherein the metal oxide comprised in the kinds of inorganic pigments composed of a spherical composite particle is independently of each other selected from the group zirconium oxide, titanium dioxide, zinc oxide or cerium oxide or mixtures thereof.

10. The method according to claim 9 wherein the metal oxide or the mixture of metal oxides is/are coated on the surface of the spherical base particle of the inorganic pigments with agglomerates.

11. The method according to claim 1 wherein the spherical base particle of the kinds of inorganic pigments composed of a spherical composite particle is independently of each other selected from magnesium silicate, aluminium silicate, alkali-metal aluminium silicates, alkaline-earth metal aluminium silicates, combinations of these silicates, silicon dioxide, glass spheres, hollow glass spheres, aluminium oxide or polymers.

12. The method according to claim 7 wherein the spherical base particles of the two kinds of inorganic pigments are the same.

13. The method according to claim 1 wherein the cosmetic formulation comprises only organic UV filters.

14. The method according to claim 1 wherein the cosmetic formulation comprises organic and inorganic UV filters.
